(19) United States
(12) Patent Application Publication
(10) Pub. No.: US 2022/0017871 A1
(43) Pub. Date: Jan. 20, 2022

(54) UNDIFFERENTIATED STATE-MAINTAINING CULTURE MEDIUM FOR PLURIPOTENT STEM CELLS

(71) Applicant: Kanto Kagaku Kabushiki Kaisha, Tokyo (JP)

(72) Inventors: Takehiro WATANABE, Tokyo (JP); Tomoya SAWAGUCHI, Tokyo (JP); Megumi YAMAZAKI, Tokyo (JP)

(21) Appl. No.: 17/299,624

(22) PCT Filed: Dec. 6, 2019
(86) PCT No.: PCT/JP2019/047857

§ 371 Date: Jun. 3, 2021

(30) Foreign Application Priority Data
Dec. 7, 2018 (JP) 2018-230014

(51) Int. Cl. C12N 5/074

(52) U.S. Cl. CPC C12N 5/0696; C12N 2533/30; C12N 2500/32; C12N 2501/04; C12N 2501/998; C12N 2501/115; C12N 2501/727; C12N 2501/33

(57) ABSTRACT

The present invention addresses the problem of providing: a culture medium that is for adherent culturing of pluripotent stem cells exhibiting high growth ability and that does not contain serum or albumin; and a highly versatile method for adherent culturing of pluripotent stem cells using the culture medium. According to the present invention, by adding, to a culture medium, a hydrophilic polymer that is conventionally known to inhibit adhesion of cells to the surface of a cell culture base material, so that the adhesion of pluripotent stem cell to the surface of the cell culture base material is enhanced, high growth ability is imparted in adherent culturing of pluripotent stem cells without adding serum or albumin thereto. In addition, by adding an antioxidant substance along with such a polymer to a culture medium, very high growth ability is imparted in adherent culturing of pluripotent stem cells.

### TECHNICAL FIELD

The present invention relates to a medium for maintaining undifferentiated state for pluripotent stem cells.

### BACKGROUND ARTS

Pluripotent stem cells such as induced pluripotent stem cells (iPS cells) and embryonic stem cells (ES cells) possess pluripotency in differentiation and replication competence, so that they have been expected to have various utilization in fields of regenerative medicine, medical research, etc., especially in regenerative medicine. Culturing of a pluripotent stem cell requires proliferating it under a safe condition while maintaining its undifferentiated state.

For conventional cell culture, cell culture media comprising serum or albumin have been used. Albumin could be either of biological origin or recombinant. There is a concern that a medium comprising albumin of biological origin might contain a virus, etc. if the doner had been infected with such virus, etc. Even when the doner is not infected with such virus, etc., it could happen that certain factor being mixed during purification of the albumin is carried in the medium, and such factor might cause the cell to differentiate at an unintended timing. In addition, because it is of biological origin, there may be lot-to-lot differences in concentrations of minor components and quality, and such differences can change the culture results. On the other hand, recombinant albumin is expensive in cost. Therefore, in culturing of a pluripotent stem cell, it is preferred to use a medium which does not comprise albumin.

In recent years, development of a serum-free medium which is suitable for culturing a pluripotent stem cell has been in progress. Patent Reference 1 discloses a serum-free medium that comprises a ligand for endothelial differentiation gene (Edg) family receptor and a ligand for serotonin receptor. Patent Reference 2 discloses a serum-free medium that comprises a Pluronic non-ionic surfactant and animal hydrolysates but that does not comprise albumin. Patent Reference 3 discloses a method for culturing an embryonic stem cell while preventing its adherence to the culture dish by using a medium that comprises polyvinyl alcohol but which does not comprise albumin. Patent Reference 4 discloses that a synthetic polymer such as polyvinyl alcohol suppresses the adherence of a cell including an embryonic stem cell to the surface of a cell-culture substrate. Patent Reference 5 discloses a medium that comprises albumin and polyvinyl alcohol.

However, there has been no medium that has a sufficient quality for a pluripotent stem cell, having a high proliferating property, being suitable for adhesive culture, etc., and having a wide applicability, without including albumin in the medium.

### PRIOR ART REFERENCES

### Patent References

### [Patent Reference 1] WO 2009/084662

### [Patent Reference 2] WO 2017/195745

### [Patent Reference 3] JPA 2007-228815

### [Patent Reference 4] JPA 2007-124982

### [Patent Reference 5] JP B 6197947

### DISCLOSURE OF INVENTION

### Problems to be Solved by the Invention

Accordingly, the present invention is aimed at providing a medium for adhesive-culturing of a pluripotent stem cell that exhibits a high proliferating property without containing serum or albumin in the medium, and a widely-applicable method for adhesive-culturing of a pluripotent stem cell using such medium.

### Means to Solve the Problems

The present inventors have searched for non-albumin ingredients that provides a pluripotent stem cell with adherence to the surface of a cell-culture substrate and found that a hydrophilic polymer increases the adherence of the pluripotent stem cell to the surface of the cell-culture substrate and that an addition of such polymer into a medium would provide a high proliferating property in adhesive culturing of the pluripotent stem cell without including albumin in the medium. The present inventors further proceeded the investigation and reached the completion of the invention.

Namely, the present invention relates to the followings:

[1] A medium for adhesive culture of a pluripotent stem cell, comprising a hydrophilic polymer but substantially no albumin.
[2] The medium according to [1], further comprising an antioxidant.
[3] The medium according to [1] or [2], wherein the hydrophilic polymer is polyvinyl alcohol or polyvinylpyrrolidone.
[4] The medium according to [2] or [3], wherein the antioxidant is at least one type selected from a group consisting of N-acetyl-L-cysteine, reduced glutathione, vitamin C, vitamin E and chlorogenic acid.
[5] The medium according to any one of [2] to [4], wherein the antioxidant is N-acetyl-L-cysteine.
[6] The medium according to any one of [1] to [5], further comprising a protein component other than albumin.
[7] The medium according to [6], wherein the protein component other than albumin is at least one type selected from a group consisting of insulin, transferrin and basic fibroblast growth factor (bFGF).
[8] The medium according to any one of [1] to [7], further comprising a differentiation suppressing agent.
[⁹] The medium according to [8], wherein the differentiation suppressing agent is GSK3β (glycogen synthase kinase 3β) inhibitor, and/or DYRK (Dual-specificity tyrosine-phosphorylation-regulated kinase) inhibitor.
[10] The medium according to [9], wherein the GSK3β inhibitor is 1-Azakenpaullone.
[11] The medium according to [9], wherein the DYRK inhibitor is ID-8.
[12] The medium according to any one of [1] to [11], further comprising NFAT (nuclear factor of activated T-cells) inhibitor.
[13] The medium according to [12], wherein the NFAT inhibitor is Tacrolimus.
[14] The medium according to any one of [1] to [13], wherein the pluripotent stem cell is derived from human.
[15] The medium according to any one of [1] to [14], wherein the pluripotent stem cell is an iPS cell.
[16] A medium for adhesive culture of a pluripotent stem cell, comprising a hydrophilic polymer, an antioxidant, and a differentiation suppressing agent consisting of a GSK3β inhibitor and a DYRK inhibitor.
[17] A method for culturing a pluripotent stem cell using the medium according to any one of [1] to [16].
[18] A method for enhancing the adherence of a pluripotent stem cell to a culture substrate, comprising adding a hydrophilic polymer into a medium.
[19] A pluripotent stem cell produced by the method according to [17] or [18].

### Effects of the Invention

According to the present invention, by including a hydrophilic polymer in the medium, a pluripotent stem cell can be cultured at a high proliferating rate while maintaining its undifferentiated state without including albumin in the medium. Furthermore, by adding an antioxidant into such medium, a higher proliferating rate can be achieved, so that a pluripotent stem cell can efficiently be cultured which is used in regenerative medicine or medical research field. Moreover, according to the method of the present invention, a method for enhancing the adherence of a cell can be provided in a medium that does not comprise albumin.

### BRIEF DESCRIPTION OF DRAWINGS

FIG. 1 shows the relevance of intracellular reactive oxygen species (ROS) level in iPS cells cultured either in albumin-added medium or in a medium with no additive in Comparative Example 1. “−” indicates the result of the culturing in the medium with no added albumin; “+Alb” indicates the result of the culturing in the albumin-added medium. The value of each result in the drawing indicates the relative value when the intracellular ROS level in the medium with no additive is considered as 100%.

FIG. 2 shows phase-contrast images of iPS cells on the third day of culturing either in the albumin-added medium or in the medium with no additive in Comparative Example 1. In the drawing, “−” indicates the image of the cells cultured in the medium to which no albumin had been added; “+Alb” indicates the image of cells cultured with added albumin. The cells take spindle morphology in good condition, whereas they take globular morphology when the condition goes wrong.

FIG. 3 shows the relationship between the antioxidant and intracellular ROS level when iPS cells were cultured either in the medium into which an antioxidant had been added or in the medium with no additive in Example 1. In the drawing, “+Alb” indicates the result of the culturing cells in a medium to which albumin had been added, “+NAC” for a medium to which N-acetyl-L-cysteine had been added, “+GSH” for a medium to which reduced glutathione had been added, “+VC” for a medium to which vitamin C had been added, “+VE” for a medium to which vitamin E had been added, and “+ChA” for a medium to which chlorogenic acid had been added. The value of each result in the drawing indicates the relative value to when the intracellular ROS level in the medium with no additive is considered as 100%.

FIG. 4 shows phase-contrast images of iPS cells cultured for 3 days either in the albumin-added medium or in the N-acetyl-L-cysteine-added medium in Example 2. “+Alb” indicates the image of the cells cultured in the medium to which albumin had been added; “+NAC” indicates the image of the cells cultured in the medium to which N-acetyl-L-cysteine had been added.

FIG. 5 shows phase-contrast microscopy images of the whole culture containers when all cells were stained blue with crystal violet in Example 3. “−” indicates the result of culturing in the medium with no additive. “+Alb” indicates the result of culturing in the albumin-added medium. “+PVA” indicates the result of culturing in the medium with polyvinyl alcohol being added. “+PVP” indicates the result of culturing in the medium with polyvinylpyrrolidone being added.

FIG. 6 shows the relative proliferating rate (mean value±standard deviation) in Example 4 when being cultured for 7 days in the basal medium alone, in the basal medium to which only N-acetyl-L-cysteine had been added, in the basal medium to which only polyvinyl alcohol had been added, or in the basal medium to which N-acetyl-L-cysteine and polyvinyl alcohol had been added. In the drawing, “basal nnedium+NAC” indicates the basal medium to which only N-acetyl-L-cysteine had been added, “basal medium+PVA” indicates the basal medium to which only polyvinyl alcohol had been added, and “basal medium+NAC+PVA” indicates the basal medium to which N-acetyl-L-cysteine and polyvinyl alcohol had been added. The numerical values on the vertical axis in the drawing refer to relative proliferating rates when that of case using only the basal medium was considered as 1.

### MODE FOR CARRYING OUT THE INVENTION

Hereinbelow, the present invention will be explained in detail based on suitable embodiments of the present invention.

The “medium for adhesive culture of a pluripotent stem cell” according to the present invention is a medium that is used for culturing a pluripotent stem cell in an adhesive manner. The “pluripotent stem cell” according to the present invention is a cell that has the pluripotency in differentiation and replication competence such that the cell is capable of differentiating into any tissue or cell that constitutes an organism. Examples include ES cells, embryonic germ cells (EG cells) and iPS cells, and iPS cells are preferred. Biological species from which the “pluripotent stem cell” according to the present invention is derived are not particularly limited, and include, for example, human, monkeys and mouse. The cell is preferably of human-origin.

The medium used in the present invention comprises a hydrophilic polymer. The present inventors have found that, in a medium that comprise substantially no albumin, the hydrophilic polymer enhances the adherence of a cell to a culture dish and promotes cell proliferation in adhesive-culturing. Although not being bound by a theory, it is assumed that the hydrophilic polymer that is added into the medium of the present invention forms a film on the surface of the cell adhered to the culture substrate, thereby increasing the adherence of the cell. Therefore, the hydrophilic polymer that is used in the present invention is not limited as long as it increases the adherence of the cell to the culture substrate. Polyvinyl alcohol, polyvinylpyrrolidone, polyethylene glycol, polypropylene glycol, polyethylene imine, carboxymethyl cellulose, methyl cellulose, dextran, gellan gum, alginic acid, etc. may be used. In view of conferring a stronger adherence to the culture substrate, polyvinyl alcohol is preferred. Moreover, the hydrophilic polymer in the present invention preferably is a non-natural product and a highly safe hydrophilic polymer that has been employed in medical products, because it is to be used in regenerative medicine and in research in medical field.

The concentration of the hydrophilic polymer that is added into the medium of the present invention is not limited as long as the hydrophilic polymer is capable of increasing the adherence of a cell to a culture substrate at that concentration, for example between 0.01% and 5%, preferably between 0.05% and 1%. In particular, polyvinyl alcohol of 0.1 to 0.5% and polyvinylpyrrolidone of 0.1 to 0.5% are further preferred in view of being capable of enhancing the adherence.

The method of enhancing the adherence of a cell to a culture substrate according to the present invention comprises adding a hydrophilic polymer into a medium, wherein the hydrophilic polymer may be added at any timing during producing or using the medium. A known culture substrate may be used according to the present invention. For example, without limitation, a culture substrate of polystyrene resin or glass may be used, with or without a protein coating such as laminin, vitronectin, collagen, gelatin or casein, or with immobilized polymer.

Since a biologically-originated albumin might have lot-to-lot differences or contamination of a factor, it is preferred not to include albumin at all, or even if albumin is included, it is preferred to be as little as possible. Therefore, in the present invention, “comprising substantially no albumin” refers to comprising no albumin at all, or comprising albumin in an extent in which it does not cause lot-to-lot difference or any influence by a factor, i.e., an extent in which it neither promote proliferation of a pluripotent stem cell nor exhibit a stability-maintaining effect. Therefore, such a medium which comprises substantially no albumin includes those comprising equal to or less than 0.010% of albumin, preferably those comprising equal to or less than 10⁻⁶% of albumin.

Known basal media may be used in the present invention, including, but not limited to, for example, DMEM (Dulbecco's modified Eagle medium), MEM (Eagle's minimum essential medium), α-MEM (Eagle's minimum essential medium α-modified type), GMEM (Glasgow minimum essential medium), Ham's F-12 (mixed nourishments F-12 Ham), DMEM/Ham, IMDM (Iscove's modified Dulbecco's medium) and DMEM/F-12 (Dulbecco's modified Eagle medium/mixed nourishments F-12 Ham). The medium preferably includes DMEM/F-12 and the like.

The components of the medium may include known additives as required. Additives may be those which do not inhibit cell proliferation, and various inorganic salts, carbohydrates, amino acids, vitamins and lipids which have conventionally been used for culturing of pluripotent stem cells may be included. Moreover, a protein component other than albumin may be included as an additive. The protein component other than albumin is, for example, a growth factor, an iron storage- or transport-factor, etc. Growth factors include, though not being limited to, epithelial growth factor (EGF), insulin-like growth factor (IGF), vascular endothelial cell growth factor (VEGF), thrombopoietin, transforming growth factor (TGF), basic fibroblast growth factor (bFGF), and insulin although it does not have a direct effect. The iron storage- or transport-factor include, though not being limited to, transferrin. More preferably, the proteins other than albumin are insulin, transferrin and bFGF. These proteins are added preferably such that the final concentration of insulin is between 1 and 50 μg/mL, the final concentration of transferrin is between 1 and 30 μg/mL, and the final concentration of bFGF is between 1 and 100 ng/mL, more preferably such that the final concentration of insulin is between 10 and 30 μg/mL, the final concentration of transferrin is between 5 and 20 μg/mL, and the final concentration of bFGF is between 2.5 and 25 ng/mL.

In one embodiment of the present invention, an antioxidant may be included as an additive. Without being bound by a theory, it is assumed that the antioxidant to be added in the medium of the present invention would contribute to a reaction in which ROS, which is produced in large amount in a cell due to the stress added to the cell, is captured and thereby is detoxified. Therefore, the “antioxidant” that is used in the present invention includes a substance that contributes to a reaction in which ROS is captured and thereby is detoxified, including, though not being limited to, N-acetyl-L-cysteine, reduced glutathione, vitamin C (L-ascorbic acid), vitamin C derivatives, vitamin E (tocopherol) and chlorogenic acid, preferably, including N-acetyl-L-cysteine and/or magnesium L-ascorbyl phosphate. The antioxidants are added preferably such that the final concentration of N-acetyl-L-cysteine is between 0.05 and 10 mM and/or the final concentration of magnesium L-ascorbyl phosphate is between 30 and 500 μg/mL. Further preferably, the antioxidants are added such that the final concentration of N-acetyl-L-cysteine is between 0.3 and 1 mM and/or the final concentration of magnesium L-ascorbyl phosphate is between 50 and 70 μg/mL.

In one embodiment of the present invention, the basal medium may comprise a differentiation suppressing agent as an additive. The differentiation suppressing agent includes, though not being limited to, GSK3β inhibitor and DYRK inhibitor, leukemia inhibitory factor (LIF), MEK inhibitor and GSK3 inhibitor. Preferably, the differentiation suppressing agents are GSK3β inhibitor and DYRK inhibitor. The GSK3β inhibitor has an effect of inhibiting the function of GSK3β or limiting its expression, and includes, though not being limited to, SB216763, BIO, AR-A014418, IM-12, CHIR99021, Kenpaullone, and 1-Azakenpaullone. The DYRK inhibitor has an effect of inhibiting the function of DYRK or limiting its expression, and includes, though not being limited to, AZ191, ID-8 and Harmine hydrochloride. Further preferably, the GSK3β inhibitor is 1-Azakenpaullone, and the DYRK inhibitor is ID-8. The differentiation suppressing agents are added preferably such that the final concentration of 1-Azakenpaullone is between 400 and 2000 nM and the final concentration of ID-8 is between 200 and 1000 nM. Further preferably, the antioxidants are added such that the final concentration of 1-Azakenpaullone is between 700 and 1400 nM and the final concentration of ID-8 is between 400 and 800 nM.

In one embodiment of the present invention, the basal medium may comprise an NFAT inhibitor. The NFAT inhibitor has an effect of inhibiting the function of NFAT or limiting its expression. The NFAT inhibitor includes, though not being limited to, Tacrolimus (FK506), Cyclosporin A, MCV-1 and INCA-6. Preferably, the NFAT inhibitor is Tacrolimus. The NFAT inhibitor is added preferably such that the final concentration of Tacrolimus is between 10 and 100 pM, further preferably such that the final concentration of Tacrolimus is between 10 and 50 pM.

The basal medium of the present invention may comprise a component other than those listed above as long as it does not inhibit the proliferation of the pluripotent stem cell. In view of efficiently expanding cells, it is selenium or ethanolamine, preferably selenium, and further preferably sodium selenite.

In one embodiment of the present invention, the medium is a medium for an adhesive culture, and it may be a basal medium comprising a hydrophilic polymer, an antioxidant, and a differentiation suppressing agent consisting of a GSK3β inhibitor and a DYRK inhibitor. The medium may further comprise known additives as needed. Adiitives may be those which do not inhibit cell proliferation, including various inorganic salts, carbohydrates, amino acids, vitamins, proteins and lipids which have conventionally been used for culturing pluripotent stem cells.

Culturing of a pluripotent stem cell using the medium of the present invention may be performed using any adhesive-culturing methods known to a skilled artisan. As an example, culturing may be performed as follows: pluripotent stem cells are washed with PBS, then cells were dissociated at 37° C. for 5 minutes to be dispersed into single-cells, collected and plated onto a laminin-coated 6-well plate for adhesive-culturing, cultured overnight at 37° C. under 5% CO₂ condition in the medium of the present invention comprising a ROCK inhibitor, and cultured for 7 days while exchanging the medium as appropriate to one that does not comprise the ROCK inhibitor.

Hereinbelow, the present invention will be explained in further detail referring to Examples, though the present invention is not limited to these Examples.

### EXAMPLES

### Comparative Example 1: Functional Validation of Albumin in Cell Culture

[Medium Preparation]

DMEM/F12 with trace element medium (from Sigma, D0547) 10.8 g, 1M HEPES (from ThermoScientific, 15630-080) 14 mL, sodium bicarbonate 1.1 g, sodium chloride 0.4 g, L-ascorbic acid phosphate magnesium salt (from FUJIFILM Wako Pure Chemical Corporation, 013-19641) 0.06 g, ITS solution (from ThermoScientific, 41400-045, Insulin 1 mg/mL, Transferrin 0.55 mg/mL, Selenium 0.7 μg/mL) 18 mL, 1-Azakenpaullone (from Toronto Research Chemicals, A800950) 361 μg, ID-8 (from Cayman Chemicals, 15222) 179 μg, and Tacrolimus (from Cayman Chemicals, 10007965) 16 ng were dissolved in 1 L of water, which were mixed to give 1 L of a basal medium.

To 100 mL of the basal medium, 1 g of albumin (from MP Biomedicals, 0215240180-100g) was added to give an albumin-added medium.

[Measurement of ROS]

In each of the basal medium with no additive and the albumin-added medium, cells were dispersed into single-cells, and after 1 hours, for each case, cells were fluorescently stained with DCFH-DA, and fluorescence intensity was measured using a flow cytometer.

[Results]

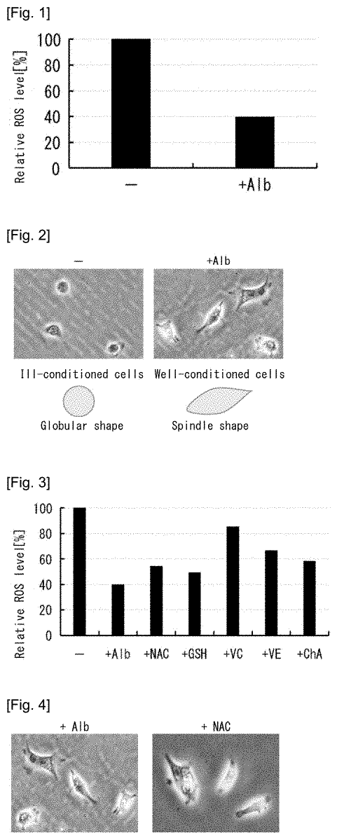

As shown in FIG. 1, it was confirmed that the intracellular ROS level was decreased by approximately 60% by albumin addition.

[Culture]

Human iPS cell line 253G1 was washed with PBS, then treated with an equivalent mixture solution of TrypLE Select (ThermoScientific) and 0.5 mM EDTA at 37° C. for 5 minutes to be dispersed into single-cells, and collected. The number of cells was counted using a hemocytometer, and then the cells were plated to be 0.25 μg/cm² onto a 6-well plate for adhesive-culturing coated with iMatrix-511 (Matrixome Inc.) at 25,000 cells/well, cultured overnight in the above-described medium comprising 10 μM Y-27632 (from Cayman Chemicals, 10005583) at 37° C. under 5% CO₂ condition, and cultured for 7 days while exchanging the medium every day to one that does not comprise Y-27632.

The culture was performed in the basal medium with no additive as a control and in the medium which is the basal medium to which albumin had been added (n=1).

[Confirmation of Cell Morphology]

The morphology of cells on the third day of culturing in the basal medium with no additive and in the albumin-added medium was observed by phase-contrast microscopy.

[Results]

As shown in FIG. 2, the cell morphology in the albumin-added medium shows a spindle shape, confirming a better condition than the cell morphology in the medium with no additive which showed a globular shape.

### Example 1: Validation of Influences of Antioxidant-Comprising Medium on Intracellular ROS Level

[Medium Preparation]

The basal medium was prepared in a similar way to in Comparative Example 1. To 100 mL of the basal medium, either N-acetyl-L-cysteine (from KANTO KAGAKU, 01763-30) 163 mg, reduced glutathione (from KANTO KAGAKU, 17501-61) 307 mg, vitamin C (from FUJIFILM Wako Pure Chemical Corporation, 013-19641) 278 mg, vitamin E (from KANTO KAGAKU, 40562-30) 430 mg, or chlorogenic acid (from KANTO KAGAKU, 10924-1A) 353 mg was added. As controls, the basal medium with no additive, and 100 mL of the basal medium to which 1 g of albumin was added were obtained.

[Measurement of ROS]

Cells were cultured in the control media or in the medium to which the respective component had been added, then dispersed into single-cells. For each case, cells were fluorescently stained with DCFH-DA, and the fluorescence intensity was measured using a flow cytometer.

[Results]

As shown in FIG. 3, a decrease in fluorescence intensity was confirmed in all of the cells cultured in the medium to which either of five types of antioxidants had been added, suggesting that the ROS level was decreased. Among the five types, a high effect was confirmed in N-acetyl-L-cysteine and reduced glutathione, confirming approximately 50% decrease in intracellular ROS level.

[Example 2: Culture of Pluripotent Stem Cell Using Antioxidant-Comprising Medium]

[Medium Preparation]

The basal medium was prepared in a similar way to in Comparative Example 1. To 100 mL of the basal medium, either N-acetyl-L-cysteine (from KANTO KAGAKU, 01763-30) 8.2 mg, reduced glutathione (from KANTO KAGAKU, 17501-61) 16 mg, vitamin C (from FUJIFILM Wako Pure Chemical Corporation, 013-19641) 14 mg, vitamin E (from KANTO KAGAKU, 40562-30) 21 mg or chlorogenic acid (from KANTO KAGAKU, 10924-1A) 17 mg was added. As controls, the basal medium with no additive, and 100 mL of the basal medium with 1 g of added albumin were obtained.

[Culture]

Human iPS cell line 253G1 was washed with PBS, then treated with an equivalent mixture solution of TrypLE Select (ThermoScientific) and 0.5 mM EDTA at 37° C. for 5 minutes to be dispersed into single-cells, and collected. The number of cells was counted using a hemocytometer, and then the cells were plated at 0.25 μg/cm² onto a 6-well plate for adhesive-culturing coated with iMatrix-511 (Matrixome Inc.) at 25,000 cells/well, cultured overnight in the above-described medium comprising 10 pM Y-27632 at 37° C. under 5% CO₂ condition, and cultured for 7 days while exchanging the medium every day to one that does not comprise Y-27632.

The culture was carried out in the basal medium with no additive and an albumin-added medium which were prepared as controls, and in a medium to which N-acetyl-L-cysteine was added, in a medium to which reduced glutathione was added, in a medium to which vitamin C was added, in a medium to which vitamin E was added or in a medium to which chlorogenic acid was added (n=1).

[Confirmation of Cell Morphology]

The morphology of cells on the third day of culturing in the control medium and in the N-acetyl-L-cysteine-added medium as a representative of 5 types of antioxidants was observed by phase-contrast microscopy.

[Results]

As shown in FIG. 4, the morphology of the cells cultured in N-acetyl-L-cysteine-added medium also shows a spindle shape, confirming a good cell condition.

### Example 3: Culture of Pluripotent Stem Cell Using Hydrophilic Polymer-Comprising Medium

[Medium Preparation]

The basal medium was prepared in a similar way to in Comparative Example 1. To 100 mL of the basal medium, either polyvinyl alcohol (from Sigma, P8136) 0.25 g, polyvinylpyrrolidone (from Sigma, PVP40) 0.25 g or 1 g of albumin was added.

[Culture]

Human iPS cell line 253G1 was washed with PBS, then treated with an equivalent mixture solution of TrypLE Select (ThermoScientific) and 0.5 mM EDTA at 37° C. for 5 minutes to be dispersed into single-cells, and collected. The number of cells was counted using a hemocytometer, and then the cells were plated at 0.25 μg/cm² onto a 6-well plate for adhesive-culturing coated with iMatrix-511 (Matrixome Inc.) at 25,000 cells/well, cultured overnight in the above-described medium comprising 10 μM Y-27632 at 37° C. under 5% CO₂ condition, and cultured for 7 days while exchanging the medium every day to one that does not comprise Y-27632.

The culture was carried out in the basal medium with no additive and an albumin-added medium prepared as controls, and in a medium to which polyvinyl alcohol and in a medium to which polyvinylpyrrolidone was added (n=3).

[Confirming Cell Attachment]

Each of the cultured cell was stained with crystal violet and the adherence of the cells was confirmed under phase-contrast microscopy.

[Results]

As shown in FIG. 5 (−), in the medium with no additive, the stained cells were confirmed only in the center of the culture container, whereas were confirmed no cell attaching on the periphery of the culture container. On the other hand, as shown in FIG. 5 (+Alb), in the albumin-added medium, stained cells were confirmed all over the culture container, confirming that there was no detachment of cell. Moreover, as shown in FIG. 5 (+PVA) and (+PVP), in cells cultured in the medium to which either of the two hydrophilic polymers was added, it was confirmed that more cells attached all over the culture container to its periphery as compared to the cells that were cultured in a condition where neither of the hydrophilic polymers was added.

### Example 4: Comparison of Proliferating Rate in Media with Each Added Components

[Medium Preparation]

The basal medium was prepared in a similar way to in Comparative Example 1.

To 100 mL of the basal medium, either only 8.2 mg of N-acetyl-L-cysteine was added, only 0.25 g of polyvinyl alcohol was added, or 8.2 mg of N-acetyl-L-cysteine and 0.25 g of polyvinyl alcohol were added.

[Culture]

Human iPS cell line 253G1 was washed with PBS, then treated with an equivalent mixture solution of TrypLE Select (ThermoScientific) and 0.5 mM EDTA at 37° C. for 5 minutes to be dispersed into single-cells, and collected. The number of cells was counted using a hemocytometer, and then the cells were plated at 0.25 μg/cm² onto a 6-well plate for adhesive-culturing coated with iMatrix-511 (Matrixome Inc.) at 25,000 cells/well, cultured overnight in the above-described medium comprising 10 pM Y-27632 at 37° C. under 5% CO₂ condition, and cultured for 7 days while exchanging the medium every day to one that does not comprise Y-27632.

The culture was carried out in the basal medium with no additive prepared as a control, and in a medium to which only N-acetyl-L-cysteine was added, in a medium to which only polyvinyl alcohol was added, or in a medium to which N-acetyl-L-cysteine and polyvinyl alcohol were added (n=3).

[Results]

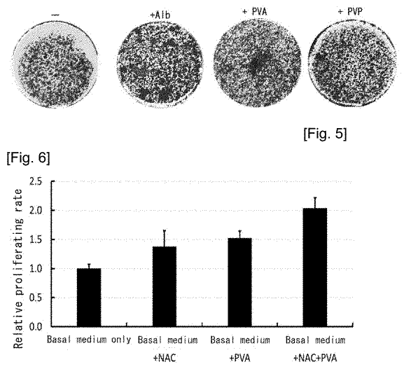

As shown in FIG. 6, approximately 1.4-fold increase in relative proliferating rate was confirmed for the case where only N-acetyl-L-cysteine was added, approximately 1.5-fold increase for the case where only polyvinyl alcohol was added, approximately 2.0-fold increase for the case where both N-acetyl-L-cysteine and polyvinyl alcohol were added, as compared to the culture result using only the basal medium.

### INDUSTRIAL APPLICABILITY

By using the medium of the present invention, it is now possible to efficiently and stably expand pluripotent stem cells without using albumin. The pluripotent stem cell cultured in such a medium can widely be used for regenerative medicine and research in medical field because it was not cultured using albumin. It is also less expensive and therefore can spread the regenerative medicine and drug development using pluripotent stem cells.

1. A medium for adhesive culture of a pluripotent stem cell, comprising a hydrophilic polymer but substantially no albumin.

2. The medium of claim 1, further comprising an antioxidant.

3. The medium of claim 1, wherein the hydrophilic polymer is polyvinyl alcohol or polyvinylpyrrolidone.

4. The medium of claim 2, wherein the antioxidant is at least one type selected from a group consisting of N-acetyl-L-cysteine, reduced glutathione, vitamin C, vitamin E and chlorogenic acid.

5. The medium of claim 2, wherein the antioxidant is N-acetyl-L-cysteine.

6. The medium of claim 1, further comprising a protein component other than albumin.

7. The medium of claim 6, wherein the protein component other than albumin is at least one type selected from a group consisting of insulin, transferrin and basic fibroblast growth factor (bFGF).

8. The medium of claim 1, further comprising a differentiation suppressing agent.

9. The medium of claim 8, wherein the differentiation suppressing agent is GSK3β (glycogen synthase kinase 3β) inhibitor, and/or DYRK (Dual-specificity tyrosine-phosphorylation-regulated kinase) inhibitor.

10. The medium of claim 9, wherein the GSK3β inhibitor is 1-Azakenpaullone.

11. The medium of claim 9, wherein the DYRK inhibitor is ID-8.

12. The medium of claim 1, further comprising NFAT (nuclear factor of activated T-cells) inhibitor.

13. The medium of claim 12, wherein the NFAT inhibitor is Tacrolimus.

14. The medium of claim 1, wherein the pluripotent stem cell is derived from human.

15. The medium of claim 1, wherein the pluripotent stem cell is an induced pluripotent stem cell (iPS cell).

16. A medium for adhesive culture of a pluripotent stem cell, comprising a hydrophilic polymer, an antioxidant, and a differentiation suppressing agent consisting of a GSK3β inhibitor and a DYRK inhibitor.

17. A method for culturing a pluripotent stem cell using the medium of claim 1.

18. A method for enhancing the adherence of a pluripotent stem cell to a culture substrate, comprising adding a hydrophilic polymer into a medium.

19. A pluripotent stem cell produced by the method of claim 17.
